(19) United States
(12) Patent Application Publication
(10) Pub. No.: US 2023/0359421 A1
(43) Pub. Date: Nov. 9, 2023

(54) SYSTEMS AND METHODS FOR ENSURING AND VERIFYING REMOTE HEALTH OR DIAGNOSTIC TEST QUALITY

(71) Applicant: EMED LABS, LLC, Miami, FL (US)

(72) Inventors: Zachary Carl Nienstedt, Wilton Manors, FL (US); Sam Miller, Hollywood, FL (US); James Thomas Heising, Richland, WA (US); John Andrew Sands, Weston, FL (US); Raymond Glenn DuBois, Palm City, FL (US)

(21) Appl. No.: 18/129,744

(22) Filed: Mar. 31, 2023

Related U.S. Application Data
(60) Provisional application No. 63/364,295, filed on May 6, 2022.
(60) Provisional application No. 63/346,761, filed on May 27, 2022.
(60) Provisional application No. 63/365,466, filed on May 27, 2022.
(60) Provisional application No. 63/366,137, filed on Jun. 9, 2022.
(60) Provisional application No. 63/366,190, filed on Jun. 10, 2022.
(60) Provisional application No. 63/487,391, filed on Feb. 28, 2023.

(51) Int. Cl. G06F 3/14; G16H 80/00; G06T 7/00; H04N 5/76

(52) U.S. Cl. CPC G06F 3/14; G16H 80/00; G06T 7/0002; H04N 5/76; G06T 2207/30168

(57) ABSTRACT

The present disclosure is directed to systems, methods, and devices are configured to ensure, improve, and/or verify the quality of the administration of a remote medical health or diagnostic exam. These systems, methods, and devices may improve the testing experience for the user and ensure test quality for the administered test. For example, the disclosure may enable easier transitioning between an augmented reality experience and pre-recorded video upon a failure with the augmented reality experience during the administration of a remote health or diagnostic exam.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application claims the benefit of U.S. Provisional Patent Application No. 63/364,295 titled “SYSTEMS AND METHODS FOR MANAGING AUGMENTED REALITY TO VIDEO TRANSITIONS” filed May 6, 2022, U.S. Provisional Patent Application No. 63/346,761 titled “SYSTEMS AND METHODS FOR RESULTS IMAGE PREPROCESSING AND RELATIVE IMAGE QUALITY VERIFICATION” filed May 27, 2022, U.S. Provisional Patent Application No. 63/365,466 titled “Continuous and Relative Identification Verification Methods for Testing and Proof-of-Result” filed May 27, 2022 U.S. Provisional Patent Application No. 63/366,137 titled “SYSTEMS AND METHODS FOR REVIEW OF ADAPTIVE VIDEO PARSING AND EVENT TAGGING” filed Jun. 9, 2022, U.S. Provisional Patent Application No. 63/366,190 titled “SYSTEMS AND METHODS FOR PROCTOR SCRIPT COMPLIANCE” filed Jun. 10, 2022, and U.S. Provisional Patent Application No. 63/487,391 titled “SYSTEMS AND METHODS FOR SYSTEMATIC TAGGING” filed Feb. 28, 2023, the entire contents of each are which are hereby incorporated herein in their entirety.

### BACKGROUND

### Field

The present application is directed to remote medical diagnostic testing and testing platforms.

### Description

The approaches described in this section are approaches that could be pursued, but not necessarily approaches that have been previously conceived or pursued. Thus, unless otherwise indicated, it should not be assumed that any of the material described in this section qualifies as prior art merely by virtue of its inclusion in this section.

Use of telehealth to deliver healthcare services has grown consistently over the last several decades and has experienced very rapid growth in the last several years. Telehealth can include the distribution of health-related services and information via electronic information and telecommunication technologies. Telehealth can allow for long distance patient and health provider contact, care, advice, reminders, education, intervention, monitoring, and remote admissions. Often, telehealth can involve the use of a user or patient's personal user device, such as a smartphone, tablet laptop, personal computer, or other device. For example, a user or patient can interact with a remotely located medical care provider using live video, audio, or text-based chat through the personal user device. Generally, such communication occurs over a network, such as a cellular, wired, or wireless network. Such communication can occur over a local network, wide area network, internet network, and so forth.

Remote or at-home healthcare testing and diagnostics can solve or alleviate some problems associated with in-person testing. For example, health insurance may not be required, travel to a testing site is avoided, and tests can be completed at a testing user's convenience. However, remote or at-home testing introduces various additional logistical and technical issues, such as guaranteeing timely test delivery to a testing user, providing test delivery from a testing user to an appropriate lab, ensuring adequate user experience, ensuring proper sample collection, ensuring test verification and integrity, providing test result reporting to appropriate authorities and medical providers, and connecting testing users with medical providers who are needed to provide guidance and/or oversight of the testing procedures remotely.

### SUMMARY

A user may administer a medical diagnostic exam using an augmented reality experience. The augmented reality experience may be mapped in a manner that timestamps various points of time, chapters, events, etc., within the augmented reality experience. In some embodiments, the system may detect an augmented reality experience failure during the user's experience. The system may identify the timestamp that corresponds to moment the system detected an augmented reality experience failure and stop displaying the augmented reality experience to the user. In some embodiments, the system may transition to presenting a pre-recorded video to the user. The pre-recorded video may be mapped similarly to the map of the augmented reality experience. In some embodiments, the system may start the presentation of the pre-recorded video at the identical or similar timestamp that the system detected the last successful augmented reality frame was presented to the user.

A system may verify image quality of an image captured by a user. The image may include a test reference card. The system may scan a code located on the reference card, which may include various test identifying elements. The system may align the image with a template by implementing feature matching. The system may assess the color of the captured image and adjust accordingly, which can help to ensure sufficient color. The system may check the detection threshold to help determine whether the image is of sufficient quality to detect a test result at or above the limit of detection. The system may further check the sharpness of the image and determine whether the image has sufficient overall quality. The system may determine the image has insufficient quality and guide the user to change the environment set up for improved image quality of subsequent images. Otherwise, the system may determine the image has sufficient quality and continue on to the interpretation of the test results.

Captured face and/or voice data can be used to create a biometric hash which can be used for continuous verification throughout an at-home diagnostic test and later as an authentication method when accessing the result. This method can, in some embodiments, allow for testing and authentication of testing results to be achieved without requiring a user to provide their license, passport, or other government-issued identification.

A system may provide adaptive video parsing and event tagging in a recording of a test session. A user may connect to a service or platform for task performance guidance, such as a proctored remote health or diagnostic testing platform, for a test session. The system may record the test session upon the connection. The system may also provide an audio, video, or audio and video connection and simultaneously save the recording data to a disk. A back-end process of the system may close the saved data file at the end of the test session and parse the data for future analysis. The system may create a database entry for the user's test session. A configured set of distributed processes within the system may query the database for accessible test sessions and begin reading the data, creating event tags and compiling the results into a project. The project may include more than one test session from more than one user. A reviewer may be presented with the project to select a test session from and review the test session. The system may load the data for the reviewer to review. The system may present to the reviewer a visual representation of the computed events of interest and a video and/or audio player. The reviewer may select a computed event of interest to review.

The system may also evaluate proctor script compliance. The system may identify words or phrases of which the proctor says during the test session. The system may then compare the words or phrases identified with a script provided by the platform prior to the start of the test session. The system may then generate a score that quantifies the percent match between the words or phrases identified and the script.

In one aspect, a computer-implemented method for managing an augmented reality user experience, the method comprising: providing, by a computer system, an augmented reality user experience to a user for use; timestamping, by a computer system, the augmented reality user experience based on at least one predetermined time interval; detecting, by the computer system, a failure of the augmented reality user experience by determining a timestamp that corresponds to the failure of the augmented reality user experience; identifying the timestamp that the failure of the augmented reality user experience occurred at; terminating, by the computer system, the augmented reality user experience at the timestamp that the failure of the augmented reality user experience occurred at; and providing, by the computer system, a prerecorded video to the user for display, the prerecorded video displaying to the user at a corresponding timestamp.

In some embodiments, the method wherein the augmented reality user experience is a remote health diagnostic exam.

In some embodiments, the method wherein the prerecorded video is timestamped based on the at least one predetermined time interval that the augmented reality user experience timestamping is based on.

In some embodiments, the method wherein the corresponding timestamp is approximately equal to the timestamp that corresponds to the failure of the augmented reality user experience.

In some embodiments, the method wherein the corresponding timestamp is less than the timestamp that the failure of the augmented reality user experience occurred at.

In another aspect, a computer-implemented method for verifying an image quality of at least one image captured by a user device, the method comprising: receiving, by a computer system, the at least one image captured by the user using a camera of the user device, the at least one image including at least one test identifying element; scanning, by the computer system, the at least one image to identify the at least one test identifying element; aligning, by the computer system, the at least one image to a template; adjusting, by the computers system, coloring of the at least one image based on a predetermined color scheme; and determining, by the computer system, whether the at least one image passes a predetermined quality threshold.

In some embodiments, the method wherein determining whether the at least one image passes the predetermined quality threshold comprises: determining whether the at least one image passes detection threshold; and determining whether the at least one image passes a sharpness threshold.

In some embodiments, the method further comprising instructing the user to capture an additional at least one image based on whether the at least one image passes the predetermined quality threshold.

In some embodiments, the method wherein the detection threshold is based on at least one of: a predetermined dynamic range; a predetermined brightness; a predetermined noise floor level; or a predetermined blur threshold.

In some embodiments, the method wherein the at least one test identifying element includes at least one of: a QR code; an NFC tag; or at least one image recognizer.

In some embodiments, the method wherein aligning the at least one image to the template includes implementing feature matching.

In another aspect, a computer system for verifying an image quality of at least one image captured by a user device, the system comprising at least one memory and at least one process, the at least one memory storing instructions that cause the processor to: receive the at least one image captured by the user using a camera of the user device, the at least one image including at least one test identifying element; scan the at least one image to identify the at least one test identifying element; align the at least one image to a template; adjust coloring of the at least one image based on a predetermined color scheme; and determine whether the at least one image passes a predetermined quality threshold.

In some embodiments, the system wherein determining whether the at least one image passes the predetermined quality threshold comprises: determining whether the at least one image passes a detection threshold; and determining whether the at least one image passes a sharpness threshold.

In some embodiments, the system wherein the at least one memory storing instruction further causes the process to instruct the user to capture an additional at least one image based on whether the at least one image passes the predetermined quality threshold.

In some embodiments, the system wherein the detection threshold is based on at least one of: a predetermined dynamic range; a predetermined brightness; a predetermined noise floor level; or a predetermined blur threshold.

In some embodiments, the system wherein the at least one memory storing instructions that cause the processor to use line detection to determine whether the at least one image passes the detection threshold.

In some embodiments, the system wherein the at least one test identifying element includes at least one of: a QR code; an NFC tag; or at least one image recognizer.

In some embodiments, the system wherein the at least one memory storing instructions that causes the processor to align the at least one image to the template includes implementation of feature matching.

In some aspects, the techniques described herein relate to a computer-implemented method for providing an augmented reality user experience to a user including: receiving, from a user device of the user by a computing system, a request for an augmented reality user experience; generating, by the computing system, data to cause the user device to display the augmented reality user experience on a display of the user device; transmitting, by the computing system, the data to the user device; timestamping, by the computing system, the augmented reality user experience based on at least one time interval; detecting, by the computing system, a failure of the augmented reality user experience; determining, by the computing system, a timestamp of the failure of the augmented reality user experience; providing, by the computing system to the user device, instructions to stop the augmented reality user experience; and providing, by the computing system to the user device, instructions to cause the user device to provide an alternative user experience to the user on the display of the user device.

In some aspects, the techniques described herein relate to a computer-implemented method, further including: providing, by the computing system to the user device, a timestamp of the alternative user experience, the timestamp indicating a timestamp of the alternative user experience at which the user device begins the alternative user experience.

In some aspects, the techniques described herein relate to a computer-implemented method, wherein the timestamp of the alternative user experience is approximately equal to the timestamp of the failure of the augmented reality user experience.

In some aspects, the techniques described herein relate to a computer-implemented method, wherein the timestamp of the alternative user experience is less than the timestamp of the failure of the augmented reality user experience.

In some aspects, the techniques described herein relate to a computer-implemented method, wherein detecting the failure of the augmented reality user experience detecting one or more of: a slowdown in network connection, a reduced frame rate, a loss of object tracking, or a frozen frame.

In some aspects, the techniques described herein relate to a computer-implemented method, wherein the augmented reality user experience includes a remote health diagnostic exam.

In some aspects, the techniques described herein relate to a computer-implemented method, wherein the at least one time interval is a predetermined time interval.

In some aspects, the techniques described herein relate to a computer-implemented method, wherein the alternative user experience includes a pre-recorded video.

In some aspects, the techniques described herein relate to a computer-implemented method, further includes: generating, by the computing system, the pre-recorded video, wherein the pre-recorded video is based at least in part on an image of an environment of the user capturing using a camera of the user device of the user.

In some aspects, the techniques described herein relate to a computer-implemented method, further including: receiving, by the computing system from the user device, an image frame; and determining, by the computing system, that the image frame passes a quality threshold.

In some aspects, the techniques described herein relate to a computer-implemented system for providing an augmented reality user experience to a user including: a non-volatile electronic storage medium, the non-volatile electronic storage medium including computer-executable instructions; and one or more processors in electronic communication with the non-volatile storage medium and configured to execute the computer-executable instructions to cause the computer-implemented system to perform steps including: receiving, from a user device of the user, a request for an augmented reality user experience; generating, data to cause the user device to display the augmented reality user experience on a display of the user device; transmitting the data to the user device; timestamping the augmented reality user experience based on at least one time interval; detecting a failure of the augmented reality user experience; determining a timestamp of the failure of the augmented reality user experience; providing, to the user device, instructions to stop the augmented reality user experience; and providing, to the user device, instructions to cause the user device to provide an alternative user experience to the user on the display of the user device.

In some aspects, the techniques described herein relate to a computer-implemented system, wherein the computer-executable instructions further cause the computer-implemented system to: provide, to the user device, a timestamp of the alternative user experience, the timestamp indicating a timestamp of the alternative user experience at which the user device begins the alternative user experience.

In some aspects, the techniques described herein relate to a computer-implemented system, wherein the timestamp of the alternative user experience is approximately equal to the timestamp of the failure of the augmented reality user experience.

In some aspects, the techniques described herein relate to a computer-implemented system, wherein the timestamp of the alternative user experience is less than the timestamp of the failure of the augmented reality user experience.

In some aspects, the techniques described herein relate to a computer-implemented system, wherein detecting the failure of the augmented reality user experience detecting one or more of: a slowdown in network connection, a reduced frame rate, a loss of object tracking, or a frozen frame.

In some aspects, the techniques described herein relate to a computer-implemented system, wherein the augmented reality user experience includes a remote health diagnostic exam.

In some aspects, the techniques described herein relate to a computer-implemented system, wherein the at least one time interval is a predetermined time interval.

In some aspects, the techniques described herein relate to a computer-implemented system, wherein the alternative user experience includes a pre-recorded video.

In some aspects, the techniques described herein relate to a computer-implemented system, wherein the computer-executable instructions further cause the computer-implemented system to: generate the pre-recorded video, wherein the pre-recorded video is based at least in part on an image of an environment of the user capturing using a camera of the user device of the user.

In some aspects, the techniques described herein relate to a computer-implemented system, wherein the computer-executable instructions further cause the computer-implemented system to: receive, from the user device, an image frame; and determine, that the image frame passes a quality threshold.

For purposes of this summary, certain aspects, advantages, and novel features of the invention are described herein. It is to be understood that not necessarily all such advantages may be achieved in accordance with the particular embodiments of the invention. Thus, for example, those skilled in the art will recognize that the invention may be embodied or carried out in a manner that achieves one advantage or group of advantages as taught herein without necessarily achieving other advantages as may be taught or suggested herein.

All of these embodiments are intended to be within the scope of the invention herein disclosed. These and other embodiments will become readily apparent to those skilled in the art from the following detailed description having reference to the attached figures, the invention not being limited to any particular disclosed embodiment(s).

### BRIEF DESCRIPTION OF THE DRAWINGS

These and other features, aspects, and advantages of the present application are described with reference to drawings of certain embodiments, which are intended to illustrate, but not limit the present disclosure. It is to be understood that the attached drawings are for the purpose of illustrating concepts disclosed in the present application and may not be to scale.

FIG. 1 is a block diagram illustrating an example protocol or method for a system that for implementing an augmented reality to video transition upon detection of an augmented reality experience failure.

FIG. 2 is a block diagram illustrating an example protocol or method for a system that preprocesses an image and verifies the image quality.

FIG. 3 illustrates an example embodiment of a captured image.

FIG. 4 illustrates an example embodiment of a scan code within the captured image.

FIG. 5 illustrates an example embodiment of an image to be aligned with a template.

FIG. 6 illustrates an example embodiment of a color reference chart within the captured image.

FIG. 7 illustrates an example embodiment of a graduated color stripe within the captured image.

FIG. 8 illustrates an example embodiment of the verification of image sharpness.

FIG. 9 is a block diagram illustrating a simplified example test process using absolute identity verification.

FIG. 10 is a block diagram illustrating a simplified example test process using relative identity verification.

FIG. 11 is a block diagram illustrating a simplified example method for showing verified proof of results.

FIG. 12 is a diagram illustrating an example of how the duration of a test can be divided into multiple overlapping time windows.

FIG. 13 is a block diagram illustrating an example of a per-window algorithm in operation.

FIG. 14 illustrates an example of a self-guided method for capturing facial recognition data.

FIG. 15 illustrates an example of a self-guided method for capturing voice recognition data.

FIG. 16 is a block diagram illustrating an example protocol or method for system that reviews a recording for adaptive video parsing and event tagging.

FIG. 17 is a block diagram illustrating an example protocol or method for a system that generates a score for proctor script compliance.

FIG. 18 is a block diagram illustrating an example protocol or method for systemic video and/or audio tagging.

FIG. 19 is a block diagram illustrating an example protocol or method for systemic video and/or audio tagging.

FIG. 20 is a block diagram illustrating an embodiment of a computer hardware system configured to run software for implementing one or more embodiments of the health testing and diagnostic systems, methods, and devices disclosed herein.

### DETAILED DESCRIPTION

Although several embodiments, examples, and illustrations are disclosed below, it will be understood by those of ordinary skill in the art that inventions described herein extend beyond the specifically disclosed embodiments, example, and illustrations and includes other uses of inventions obvious modifications and equivalents thereof. Embodiments of the inventions are described with reference to accompanying figures, wherein like numerals refer to the like elements throughout. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

As mentioned briefly above and as will now be explained in more detail below with reference to the example embodiments provided in the figures, this application describes systems, methods, and devices that are configured to ensure, improve, and/or verify the quality of the administration of a remote medical health or diagnostic exam. Embodiments of the inventions described herein can comprise several novel features and no single feature is solely responsible for the desirable attributes or is essential to practicing the inventions described.

Accuracy of diagnostic or health test results interpretation can be important. If more useful health test data can be collected and/or if an improvement in the confidence of proper administration of the health or diagnostic test can be made, then the results of the health or diagnostic test can be improved and better utilized. Proctored or certified remote diagnostic testing may rely on cameras or sensors of a user device to relay images, video, audio, or other information to proctors or other systems for viewing, listening, processing, and so forth. Test results interpreted by a user, under guidance of a proctor or not, can be influenced by human error. One effort to minimize human error can include minimizing distractions and frustrations during the administration of a medical diagnostic exam, which may contribute to more accurate results interpretation of the medical diagnostic exam.

Similarly, it may be critical to know that the person presenting, using, or relying on the test result is the same person that took the test. This may especially be true when it comes to remote, at-home, and other unsupervised or supervised diagnostic self-testing methods where supervision may be absent or limited because the supervisor (e.g., proctor) is not physically present with the test taker or in situations where a test taker may have an incentive to misrepresent the results of their test. For example, there are a variety of scenarios in which public health concerns may require a positive or negative test result for an infectious disease in order for an individual to engage in some activity. A scenario may include when a negative test result for an infectious disease is required to board an airplane or to gain entry to a place where risk of spreading infection is high. A positive test result for a particular disease may be required to obtain an appropriate medication or other treatment. As another example, an employee or prospective employee who is required to take a drug test may have an incentive to interfere with normal testing procedures. Thus, there is a need for a method to ensure, verify, or improve the overall remote testing quality. This can include, for example, authenticating self-testing procedures and results that are resistant to a malicious individual or accomplice trying to pose as the user, providing a system that can timestamp an augmented reality experience and efficiently degrade to a presentation of a pre-recorded video or other testing experience, such as images, illustrations, text, audio, and so forth. upon detecting an augmented reality experience failure and providing a system that can ensure sufficient quality of images received from a user device so that computer vision-based results interpretation can be used to interpret test results more accurately.

### Augmented Reality Experience for Test Administration

In some instances, a user may view an augmented reality experience. The augmented reality experience may be used in conjunction with the administration of a medical exam, health or diagnostic test, or the like. The augmented reality experience can be facilitated with the use of a user device (such as a cellphone, smartphone, tablet, laptop, personal digital assistant (PDA), or the like). For example, using the user device, the user may be presented with an augmented reality experience that guides the user through self-administration of the diagnostic test. A system, such as a remote healthcare or proctoring platform that utilizes, facilitates, or provides the augmented reality experience, may timestamp, or map the augmented reality experience provided to the user, for example by tracking which step of a testing procedure the user is currently at, which steps have been completed, which steps remain, and so forth. The timestamp of the augmented reality experience may function as a marker for the augmented reality experience, which can divide the augmented reality experience into readily identifiable moments of time, events, chapters, etc. For example, an event or chapter can be a particular step or series of steps in a testing procedure. The augmented reality experience can be displayed to the user and the user can experience the augmented reality as the execution of the augmented reality experience proceeds through the map of the augmented reality experience. In some instances, the augmented reality experience may be executed until an augmented reality experience failure is detected by the system. An augmented reality experience failure may include a glitch in the experience, a slow down of the experience, a decreased frame rate of the experience, a frozen frame within the experience or display, loss of object tracking (e.g., loss of the user face tracking, loss of test kit component tracking), etc. The failure may occur prior to the user completing the augmented reality experience, which may hinder the overall experience of the user due to frustration, wasted time, inability to complete a diagnostic test associated with the experience, etc.

In some instances, the user device can be in communication with a system, such as a remote testing platform. Upon the system detecting an augmented reality experience failure, the system may identify the timestamp of the last successfully displayed frame of the augmented reality experience presented to the user. The identification of the last successfully displayed frame may pinpoint the time at which the user was no longer able to receive or experience augmented reality. The system may detect an augmented reality experience based on any of a number of different factors or based on a combination of different factors. Such factors can include analysis of a live video stream received from the user device, user device performance, network connectivity, input from a proctor associated with the system, etc.

In some embodiments, the augmented reality experience can be provided utilizing a system other than the user's device. For example, the user device can be in communication with a system as described above. The system can be configured to communicate with the user device for various purposes. For example, the system can indicate steps to be performed, can process video received from the user device to identify features, provide augmented reality overlays, and so forth. In the event of a network disruption (e.g., a slowdown in network performance, intermittent connectivity, and so forth), the system can adjust the augmented reality experience, for example by terminating the augmented reality experience and switching to a pre-recorded video, still images, text, audio, and so forth. In some embodiments, the type of experience can be dynamically selected based on the performance of the network connection. For example, if the user's network connection has slowed down but is still sufficiently stable and fast for a video experience, the system can switch to a pre-recorded video. If the user's network connection is very slow or intermittent, a less network-intensive experience can be selected.

In some embodiments, the augmented reality experience can occur on the user device. For example, the augmented reality experience can be performed locally without the need for a network connection. Accordingly, in some embodiments, an augmented reality experience can continue even if the user device's network connection suffers degradation or disruption. The user device can switch to a different experience (e.g., pre-recorded video, images, audio, text, etc.) upon detecting an issue with the augmented reality experience.

In some embodiments, a pre-recorded video can mimic the augmented reality experience. The pre-recorded video may be a video version of the augmented reality experience. This may keep the user's experience with the system progressing forward and prevent the system from wasting the user's time in the case of an augmented reality failure. The pre-recorded video may be mapped or timestamped in a similar manner to the map or timestamps of the augmented reality experience. For example, an event that may be mapped or timestamped within the augmented reality experience may also be mapped or timestamped within the pre-recorded video. The transition of the system from displaying the augmented reality experience to displaying the pre-recorded video may be manipulated to blend the transition in a manner that may prevent the user from recognizing the failure of the augmented reality experience. For example, the transition may appear as a blurry intermediate frame or a frame that appears as though the experience is in a buffering or loading state. In some instances, the virtual content may be warped using intermediate computer-generated frames between the augmented reality experience and the pre-recorded video to provide an appearance that the augmented reality virtual content is moving toward the identical location of where it may be presented in the pre-recorded video. This may lead to the prevention of an abrupt or startling transition between the augmented reality experience and the pre-recorded video. The display of the pre-recorded video may begin at the pre-recorded video frame that is similar or identical to the augmented reality frame that is identified as the last successfully displayed augmented reality frame presented to the user. The timestamp of the last successfully displayed augmented reality frame presented to the user can be similar or identical to the timestamp of the pre-recorded video that the system begins to display the pre-recorded video to the user. This may provide a seamless experience for the user in the event that an augmented reality failure may be detected by the system.

In some instances, the timestamp mapped within the augmented reality experience may correspond to a chapter mapped within the augmented reality experience. Upon the system detecting an augmented reality experience failure, the system may transition to the pre-recorded video, beginning the display of the pre-recorded video at the beginning of the chapter that corresponds to the time at which the augmented reality experience failure was detected. This may provide the user an opportunity to review any part of the experience the user may have been distracted during.

In some instances, the pre-recorded video may resemble the environment in which the user may be administering the augmented reality experience. For example, the user may capture a photograph with the user's personal device that demonstrates that background of which the augmented reality experience may take place. The photograph may be used to create a custom pre-recorded video which may allow the video to display content on top of the user's background. This may further disguise the transition between the augmented reality experience and the presentation of the pre-recorded video.

While as described above, the pre-recorded video can be adapted to mimic an augmented reality experience. In some embodiments, the pre-recorded video may not mimic the augmented reality experience. For example, a user device or system (e.g., telehealth platform) can switch to a pre-recorded video showing steps to be completed. Such an approach may be preferable if a user is using a relatively low-powered device that may struggle to generate a custom pre-recorded video due to a lack of processing power, limited battery capacity, and so forth. A system such as a telehealth platform may have insufficient resources to quickly generate custom pre-recorded videos, particularly during times of high platform demand.

### Image Quality for Test Administration

In some instances, a remote health or diagnostic test session may require the user to perform steps or actions throughout the administration of the test session. These steps or actions can be shown or exemplified throughout the augmented reality experience and/or pre-recorded video. For example, a user can capture an image of a reference card that may be used for administration of a medical exam, health diagnostic test, or the like. Such diagnostic or health test can include a diagnostic test for COVID-19, influenza, urinary tract infections (“UTI”), sexually transmitted infections, drugs, or other diagnostic tests. The reference card may contain test identifying elements and/or other elements that can be used in test interpretation, such as a code, a results strip, a color gradient, etc. The code may include a QR code, NFC tags, image recognizers (e.g., images, patterns, etc., that can be used to identify the test), etc. The results strip may be seen in the image in front of the reference card. Various regions of the reference card can be used to preprocess the image and verify the image quality. For example, an image can be preprocessed prior to results interpretation.

In some instances, for example, to ensure, verify, or improve test quality, the quality of the image captured may be assessed. To assess the image quality and/or preprocess the image, a system may scan or interpret the code within the captured image. In some embodiments, the code can include a unique ID, which may be different for each reference card manufactured and can allow each reference card to be individually recognized or individually identified. In some instances, the unique ID can be used to track the diagnostic or health test throughout the testing process, which may mitigate risk of fraud or error. Additional information may be contained within the unique ID, or the unique ID can be used to retrieve additional information from a database or other data store. For example, a manufacturing lot number, an expiration date of the test, the test type, the test version, and so forth can be determined, either directly from the code or by querying a database or other data store. Much of the information contained within the unique ID or linked to the unique ID (e.g., in a database or other data store) can be used to further differentiate the test from others. For example, the manufacturing lot number can be used to track manufacturing defects. Similarly, for example, the expiration date can be used to validate the efficacy of the test prior to administration of the test. As another example, the unique ID can be used to determine whether a test with the same unique ID has already been taken, which can indicate an attempt to commit fraud and/or to reuse a test. As another example, the test type and test version can be found in a database to determine where various reference card features, test strip identifiers or lines, and so forth should appear.

Additionally, in some instances, for example, the system may align the captured image to match a template. Alignment of the image to the template allows the system to be able to register which pixel regions to look at for the various features on the reference card. In some instances, the system can implement feature matching to align the image, which may be an effective method to align the image. For example, the features of the reference card can be used to obtain a geometric transform that aligns the captured image with the template, for example by minimizing a mean squared error, mean absolute error, and so forth. In some embodiments, the captured image can be considered to be aligned when the difference between the captured image and the template is below a threshold value. In some instances, after alignment of the image and the template, fixed pixel range addresses can be used to access the other information on the reference card. This may also improve further processing, manual or automatic, by ensuring every test image can be standardized in orientation, resolution, and so forth.

Additionally, in some instances, for example, the system can correct the color of the captured image. The captured image of the reference card can include a color reference chart, which may include printed known colors. By measuring the colors of one or more areas of the captured image, the system can adjust the image to represent “true” or “reference” color(s) for the test strip. In some instances, if the color of the image is not corrected, the test strip elements or test strip stripes may have a different color profile than may be expected due to the type of lighting in the environment where the image was captured, the white balance of the camera, and so forth. In some instances, restoring the “true” or “reference” color can improve the accuracy of manual or automatic interpretation of results by normalizing the appearance of images.

Additionally, in some instances, for example, the system can check whether an image is of a sufficient quality to detect a test result at or above the limit of detection. For example, a reference card can include graduated color strips. The system may evaluate the graduated color stripes that may be located on the test strip or reference card to determine a detection threshold. In some instances, the lightest or faintest graduated color stripe can be printed on the reference card at an intensity that may be slightly below the desired detection threshold for the actual test strip. By treating these lines as “results lines” and attempting to detect the “results lines,” the system can assess whether the image has sufficient dynamic range, contrast, brightness, noise floor, etc., to be able to detect a “weak positive” result that may be on the actual test strip. The system may use region of interest, pixel region averaging, color isolation, deep learning, etc., to determine whether the image is of sufficient quality to detect a test result at or above the limit of detection. This may be useful in maximizing the accuracy of manual or automated results interpretation.

In some embodiments, the system may determine the sharpness of the captured image. In some instances, the camera used to capture the image may utilize an autofocus feature of the camera to sharpen the image to be captured. However, such features can still result in blurry or fuzzy images due to, for example, poor lighting, focus on a different object in the field of view of the camera, and so forth. The system can help to ensure the image captured has sufficient sharpness. For example, the system can check various regions of the image (e.g., QR code, company logo, fiducials, edges, etc.) to measure blur. The system may reject images that are too blurry. For example, the system may reject an image in which features that are expected to be sharp are spread over too many pixels, when transitions from one color to another occur over too many pixels, and so forth. For example, If a logo is printed in black on a white card, a sharp transition from black to white can be expected at the edges of the logo. If the transition is not sudden, this may indicate that the image is too blurry. In some instances, a blurry image can cause edges to expand and may impact the appearance of results lines. In some instances, the interpretation of the results after administration of a diagnostic test may be improved by both manual and automatic means when the system can ensure a sharp image.

In some embodiments, an image may pass the quality check upon the completion of the previous corrections and checkpoints, such as scan code, align image, correct image color, check detection threshold, check image sharpness, etc. In some instances, corrections or checkpoints may be completed simultaneously. The system may determine at any point along the image quality verification process that the image may not be of sufficient quality and direct a user to collect an additional image. In some embodiments, the system can direct the user to change the setup of the image capture, for example by increasing lighting, moving closer to the object to be imaged (e.g., a reference card), and so forth.

In some instances, if the image quality is not sufficient, the user may be guided to change the setup for improved image quality. For example, the user may be guided to improve the user's environment so that the next image captured may be of more sufficient quality. Various variables may affect the adequacy of the user's environment such as brightness, lighting location, shadows, dirty camera lens, etc. For example, more light in the room may reduce noise and improve image quality, a bright light source in front of or directly behind the reference card may degrade the image, an overhead light or indirect lighting may enhance the image, shadows from nearby objects may also affect image quality, a dirty camera lens may degrade or blur the image, etc. In some instances, the system may recognize various types of lighting environments and may assess the image and intelligently suggest the best alteration of the environment to the user, along with graphics or animated instructions. In some instances, the system may prompt the user with instructions such as, for example, “Please turn on an additional light or move to a brighter place,” “Please turn the test around so that the nearest window is behind you,” “Please clear the area around the test of any large objects,” “Please gently wipe off your camera lens,” etc.

### User Identify Verification for Test Administration

To further ensure, verify, and/or improve test quality, continuous or periodic biometric identity verification can help to ensure that a single individual completes a diagnostic test without substituting an accomplice or other individual, and does so in a manner that does not negatively impact the test time or user experience. This identity verification also makes sure that when a test result is provided, the person presenting the result is the same as the person who took the test. This can prevent test takers from sharing their results with others or having their results used without their knowledge.

User continuity can be monitored by the creation of a unique biometric hash. A video recording of a user's face and/or an audio recording of their voice can be run through an algorithm to create a unique hash for that user. Thereafter, an arbitrary video and/or audio recording can be compared to the hash in order to analyze whether or not it belongs to the same user. This comparison and analysis can be performed on a continuous stream of data, on periodically received data, and so forth.

A biometric hash can be used for continuous user identity verification during a test in a variety of different methods.

In some instances, a user can connect to a service or platform which may guide the user through the performance of a task. The service or platform may include a service or platform for use with administration of a remote health or diagnostic test. The service or platform may include a proctor that may guide the user through the administration of the health or diagnostic test. In some instances, a system may record the user's test session that includes the administration of the diagnostic or health test. During the test session for the administration of the health or diagnostic test, a test proctoring system may request for the proctor to provide input as to when key events or events of interest may have occurred. The input may be provided by the proctor or by a computer-based review, in which the events may include computed events of interest. The key events may be predetermined, such as the scanning of a test kit expiration date, user identification card view, the swabbing of the user's nostrils, opening packaging, results interpretation, etc. The proctor input may create a tag, or a timestamp associated with the key event, which can help with later video queries related to the key events. For example, during the test session, a proctor may indicate (e.g., by computerized indication) when the user may be performing certain steps that may be involved in collecting a test sample which can include a key event. In some instances, the computer-based review may identify key events or computed events of interest for which there may not be an associated proctor input request. In identifying key events, the number or length of video clips from the test session that may be analyzed in review may be reduced or minimized.

This may be useful, for example, to review a testing session that has received a poor rating from a user. In an example, a user has provided a 2-star review of their user experience with their test session. A recording of the user's test session may be reviewed to identify generally, or specifically, when or at which point during the testing session a negative user experience, or a negative user emotion may have occurred. The identification of the negative user experience or negative user emotion may become a key event or an event of interest.

In some instances, a computer-based review or search for key events may be based on a variety of parameters. For example, the parameters may include recognition of words, phrases, or other vocalizations or expression captured within the recording. In some instances, an audio, video, or video and audio connection may be made to the system upon the user connecting to the service or platform to begin the administration of the health or diagnostic test. The data associated with the recording of the test session that includes audio, video, or video and audio connection may be saved in a more efficient manner than by size or by time limit to help ensure the number of words or phrases cut-off or inadequately captured can be minimized. Such efficient manners may initially include identifying key events within a test session.

In some instances, the system may save and store the audio, video, and/or audio and video data associated with the recording of the test session. For example, computer-based review of the test session data may include the identification of key events, such as moments, words, phrases, movements, test steps, etc. within a test session. This may help to identify and study key events that affect a user's experience. The key events may be placed into a data store or table that can include location information associated with the key events. For example, the location information can include a timestamp, chunk number, test session information, etc. This may provide easier access to open and review the key events.

In some instances, the system may close the saved data files, which include audio, video, and/or audio and video data, at the end of the test session. The system may break or parse the recording into chunks such that the beginning and end points of the chunk may occur when neither the user nor the proctor may be speaking. In some embodiments, visual cues may be avoided during the dynamic chunking, such as showing a user identification, swabbing of the user's nostrils, etc. This may help a reviewer of the recording at a later time by helping to enable the reviewer to select various key events or times of interest within the recording, rather than scrub through or review the entirety of the recording. In some embodiments, speech recognition analysis may be requested. This may employ an algorithm that may use signal processing to create dynamically sized chunks, which may be based on breaks in speech patterns. For example, in some embodiments, a chunk can end only after a sound level is below a threshold amount for at least a threshold amount of time. This may help to ensure a single word, sentence, etc., may not be separated between two video or audio files.

In some instances, a database entry may be created for a user's test session. The database entry may include recording metadata and key events. In some instances, a configured set of distributed processes may query the database for accessible sessions and may begin reading data and creating events which may include key events. In some instances, the events may be created while the test session may still be live or ongoing. In some embodiments, events can be created by the user device, for example by a script running in the user's browser, an application running on the user's user device, and so forth. For example, such events may include recognizing key spoken words or utterances from audio, the closing of the user's eyes for an extended period of time, faces entering or leaving a camera view, shifts in characterized emotional state (e.g., face shape, smile, frown, eyebrow raise, etc.), voice timbre, interactions between the user and the proctor, etc. In some embodiments, natural language processing, sentiment analysis methods, speech-to-text technology, object recognition technology, and so forth can be used to identify events.

In some instances, a reviewer may be presented with a project to review. The project may include more than one test session which may be from more than one user, multiple test sessions from a same user, and so forth. In some embodiments, the reviewer can select a session to review based on a unique identifier associated with the session.

In some instances, the audio, video, and/or audio and video recording data may be loaded for the reviewer to review. The reviewer may see a visual representation of the computed events of interest, or key events, as well as a video and/or audio player. The reviewer may be able to click on or otherwise select an event which may signal the player to send the recording to or near (e.g., approximately one to two seconds prior to) the event so the reviewer can hear and/or view the event and perform any number of actions which their review may require.

In some instances, a proctor may be provided with a script to use to guide the user during a testing session. The script may include instructions or signals for the proctor to use for guidance during the test session. The script may be continuously or iteratively updated over time to improve the user's experience, clarity, or compliance with regulation or best practice standards. In some instances, some proctors may stick to a current version of the script, whereas other proctors may use an outdated version of the script. In some instances, some proctors may go off script in which they may say words or phrases during the test session that are not included in the script. Each approach may have individual benefits for the user to optimize their testing experience.

For example, a proctor that may use a current version of the script may benefit from the ongoing iterative edits and updates to the script that may have been made in consideration of user satisfaction surveys, user reviews, user ratings, quality assurance measures, etc. In some instances, allowing the proctor to go off script in certain situations may also improve the user's experience. The user may be confused or may need specific or additional instructions regarding a particular step in the test session. The user may also ask unique or specific questions to the proctor during the test session, which may lead the proctor to go off the script provided to the proctor to address the specific needs of the user.

In some instances, script compliance can be evaluated by first identifying words or phrases the proctor says. For example, the words or phrases the proctor says can be identified using the method described above (e.g., text-to-speech, natural language processing, etc.). The words or phrases identified by the system can then be compared to a current version of the script. In some instances, the comparison can result in a score that may quantify the percent match between the words or phrases the proctor said and the current version of the script. In some embodiments, a scoring metric may not allow deviations from the script. In some embodiments, a scoring metric can allow the proctor to deviate from the script. In some embodiments, a scoring metric can allow the proctor to deviate from the script while requiring compliance with some parts of the script. For example, a scoring metric may be used that may not allow for additional words that may be off script to affect the evaluation of the proctor or count against the proctor's score. For example, extra information may be provided by the proctor to a user in a specific situation and the extra information said may not affect the evaluation of the proctor when the proctor's words are compared to a current version of the script.

In some instances, a cumulative average can be determined for each proctor in addition to each proctor's individual score which may help to determine trends for each proctor. In some instances, a cumulative average can be determined for each proctor team which may help to determine overall trends. The proctor teams can be internal or external teams. For example, a trend that may be determined may be the determination of how quickly each proctor or proctor team adapts to an updated script. In some instances, identifying which sessions or proctor teams may most often be using outdated scripts can be useful in providing targeted education regarding script changes. In some instances, a proctor team may be slower to adjust to the updates which may slow the user experience improvement or negatively impact the user experience. In some instances, additional proctor education or emphasis on script adherence may be recommended for such proctors or proctor teams. In some instances, users may not be directed or assigned to a proctor or a proctor team that may be using outdated scripts, that have an unsatisfactory evaluation associated with them, and so forth under various conditions. Such conditions may include, for example, whether the user may be considered a new user, whether the number of users requesting a test session at a time is low, whether the proctor or proctor team has shown improvement (for example, an increase in a compliance metric at or above a threshold value), etc.

Reviewing a user's test session that includes the administration of a health or diagnostic test to study specific aspects of a user's test experience can be important in ensuring test integrity, monitoring proctor performance, providing training or quality assurance, etc. Similarly, positive interactions, negative interactions, potential test security issues, test session abnormalities, technical difficulties, test instruction compliance, proctor instruction delivery, identification of user actions or specific test kit components, suspicious user activity, user activity that may be nonconforming to the test session, etc., can also be reviewed and possibly improved upon with review. Proctor-dependent tagging of user suspicious or nonconforming activity may help to identify a segment during the test session that should not be considered when determining the test result, that should lead to invalidation of the test result, and so forth. However, proctor-dependent identification of key events or events of interest may also be unreliable because a timestamp associated with the proctor's input may not align with the portion of the test session recording where the event may have actually occurred. This mismatch may make a retrospective review of the test session difficult and may result in slow, inefficient, and tedious searching to identify a correct portion of the recording for review. Accordingly, it may be beneficial to implement a computer-based review system that can identify and tag key events to be reviewed or extracted at a later time and provide easier access to the key events upon review. Such a computer-based review system can make use of technologies such as voice-to-text, sentiment analysis, and so forth. In some embodiments, tags can be created in response to a user performing particular steps more slowly than expected, not making progress on the test, completing steps too quickly, and so forth.

In some embodiments, data produced during a test session can be important for determining a health or diagnostic test result, determining whether such a result is valid, and so forth. To capture or collect this data, the proctor and/or the processor of the system may tag the video and/or audio produced from the health or diagnostic test session continuously or periodically while the test session is being administered. For example, the proctor may take action to tag the video and/or audio produced from the health or diagnostic test session. The tag may be related to an instance in which the user may be acting in a way that may not be in compliance with the health or diagnostic test. This activity may include the user acting suspicious with their test device. For example, a suspicious act the user may undertake may be trying to add an unknown liquid that was not included in the test kit. In some embodiments, the activity may also include the user coughing, sneezing, reflexing, or any activity of the like. The activity can include activity related to fraud. For example, if there is an instance during the administration of the health or diagnostic test in which the user cannot be seen or heard during the test session, if there is more than one face seen in the video during the test session, or if there is more than one voice heard in the audio during the test session, the activity may be tagged. In this manner, the proctor may tag the video and/or audio produced, which may allow the system to record the specific time and context of the suspicious activity. The context may be related to a step in the health or diagnostic test when the suspicious activity occurred. In some embodiments, the proctor, health care practitioner, or the like, may review the tagged data at a later time. In some instances, the tagged data may be removed or extracted from the data prior to the data being sent to a health care provider for review and determination of the result of health or diagnostic test. In some embodiments, the tagged data can be flagged for review by a health care provider, proctor, or the like, for example to determine if a result should be invalidated due to abnormalities during the administration of the health or diagnostic test.

In some instances, the system may automatically tag the video and/or audio produced from the health or diagnostic test session. In this manner, a processor that may process the test session during the administration of the health or diagnostic test may generate a tag if the processor identifies an instance in which the user may be acting in a way that may not be called for in the health or diagnostic test as described above.

In some instances, the tag may be related to other data points, events, or occurrences during the administration of the health or diagnostic test. This may include coughing, blinking, emotion from facial expression, emotional voice, tone of voice, identifiable key words, etc. Tagging the video and/or audio produced from the health or diagnostic test session may allow for the remote health or diagnostic test session to be able to provide a similar level of confidence in the test results for the user and/or health care provider. This may allow for the remote administration of the health or diagnostic test to better replicate the administration of the same health or diagnostic test in person.

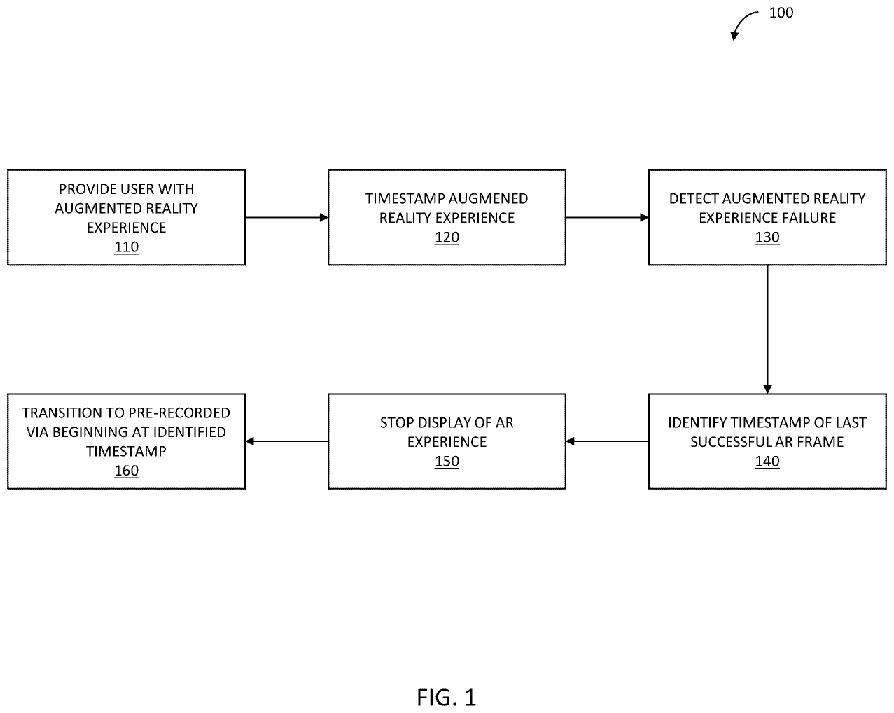

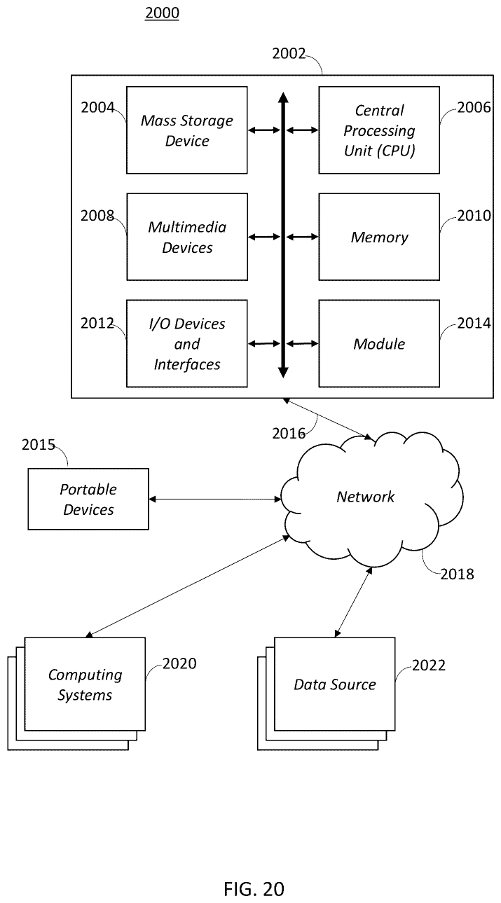

FIG. 1 illustrates a block diagram of an example protocol or method 100 for implementing an augmented reality to video transition upon detecting an augmented reality experience failure. The method 100 can be implemented, for example, using one or more components of the system shown in FIG. 20. As discussed above, the method 100 can take place wholly or partially on a user device and/or another system, for example a system associated with a telehealth provider.

At block 110, a user may participate in or view an augmented reality experience. As described above, the augmented reality experience may be used in conjunction with the administration of a medical diagnostic exam. At block 120, a system may map the augmented reality experience by identifying various moments of time, chapters, events, etc., and timestamping those identified.

At block 130, the system may detect an augmented reality experience failure. This may be a point at which the user may no longer be able to experience augmented reality or a point at which the user may no longer be able to progress through the augmented reality experience. Upon detecting the augmented reality failure, with the use of the map, the system may identify the timestamp of the last successful augmented reality frame presented to the user. At block 150, the system may stop displaying the augmented reality experience to the user.

At block 160, the system may transition from displaying the augmented reality experience to the user to presenting a pre-recorded video to the user. The pre-recorded video may begin to play at the identical timestamp that the last successful augmented reality frame was displayed to the user or at a timestamp close to the timestamp when the last successful augmented reality frame was displayed to the user (e.g., within about one second, within about two seconds, without about five seconds, within about ten seconds, etc.)—. This may ensure the user's use of the experience is not hindered.

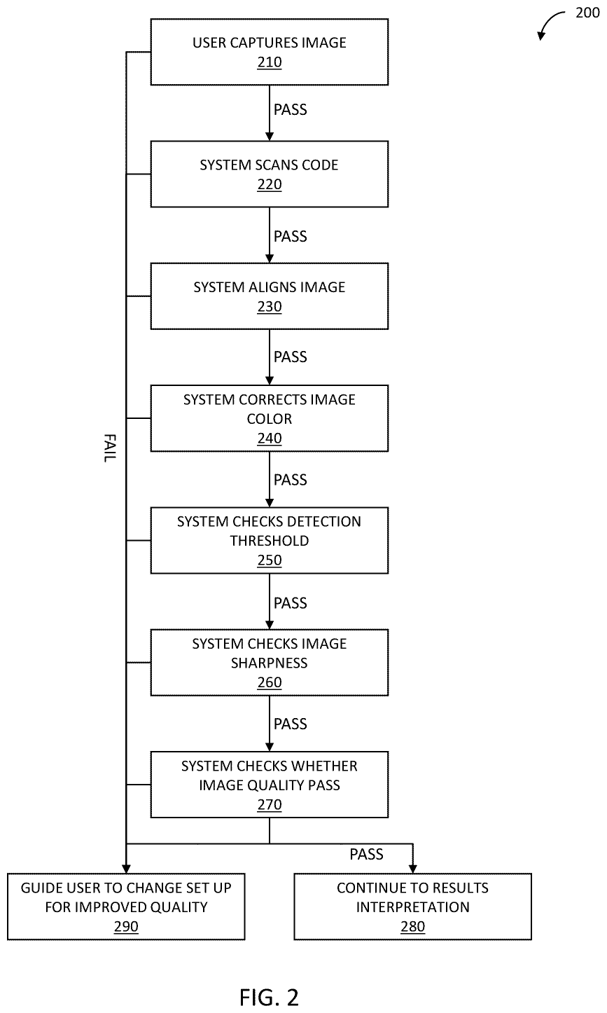

FIG. 2 illustrates a block diagram of an example protocol or method 200 for a system that preprocesses an image and verifies the image quality. The method 200 can be implemented, for example, using one or more components of the system shown in FIG. 20.

At block 210, a user may capture an image. The captured image may be of a reference card, which contains test identifying elements. At block 220, a system may scan a code that may be located on the reference card. The code can include various information about the test, for example, a unique ID, manufacturing lot number, expiration date, test type, test version, etc.

At block 230, the system may align the image with a template. This can allow the system to recognize which pixel regions to look at for the various features on the reference card that may be used in the follow steps. At block 240, the system may correct the color of the image by measuring the color that the camera reports for the different squares in the region of interest and adjusting the color accordingly, for example based on known reference values.

At block 250, the system may determine whether the image may be of sufficient quality to detect a test result at or above the limit of detection. The system may assess whether the image has sufficient dynamic range, brightness, and noise floor that may be necessary to detect a result on a test strip used to administer a test. The system may use a line detection algorithm to make this determination.

At block 260, the system may check the sharpness of the image by checking various parts of the image to measure blur. The system may reject images that have a blur that may be too large as measured in pixels. At block 270, or at any step within the method, the system may determine whether the image may pass the quality check. If the image passes the quality check, at block 280, the system may continue to results interpretation. If the image does not pass the quality check, at block 290, the system may guide the user to change its setup which may improve the quality of a subsequently captured image.

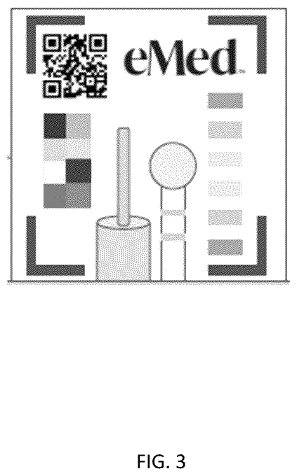

FIG. 3 illustrates an example embodiment of a captured image. The captured image may be of a reference card, which contains test identifying elements. Various regions of the reference card can be used to complete the image quality verification process.

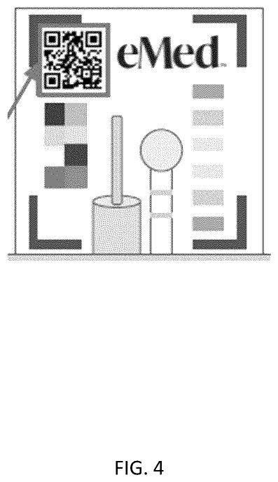

FIG. 4 illustrates an example embodiment of a code that a system may scan to access various information. The code may include a QR code, NFC tags, image recognizers, etc.

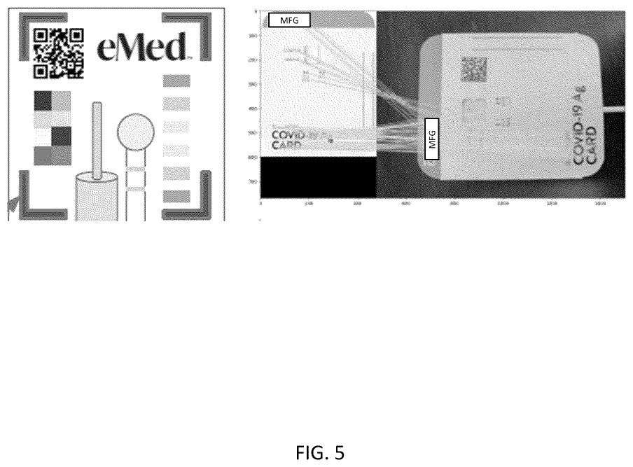

FIG. 5 illustrates an example embodiment of aligning a captured image with a template. The system may align the captured image with a template using various algorithms, such as SIFT. The system may implement feature matching to align the image with the template.

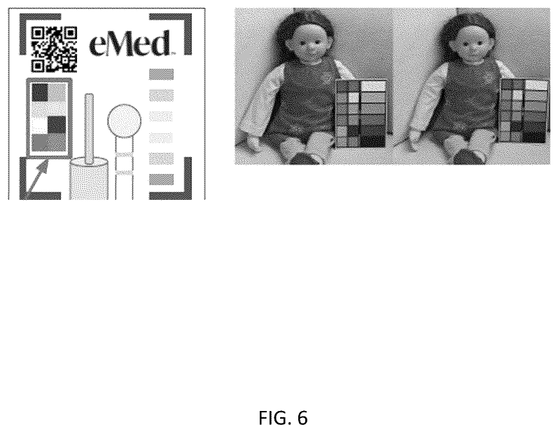

FIG. 6 illustrates an example embodiment of a reference card that may include a color reference chart. The chart may contain printed known colors that may serve as color references for a test strip.

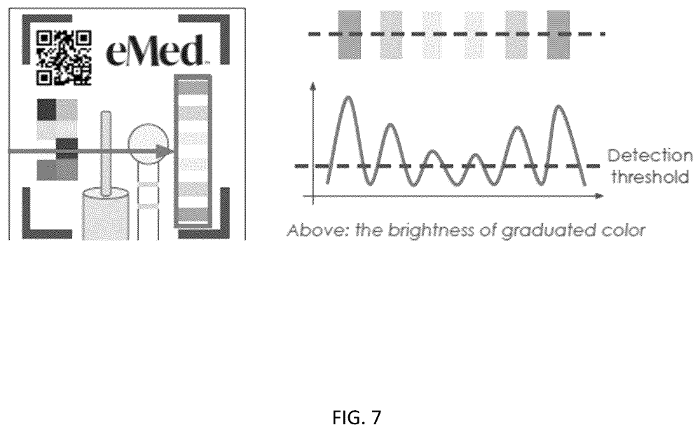

FIG. 7 illustrates an example embodiment of a reference card that may include a graduated color stripe printed on the reference card. The graduated color stripe may be printed on the reference card at an intensity slightly below the desired detection threshold for the test strip used to administer the test. The graduated color stripe may be used as “results lines” for the image quality verification process. The brightness of graduated color stripes can be plotted, and the detection threshold may be determined.

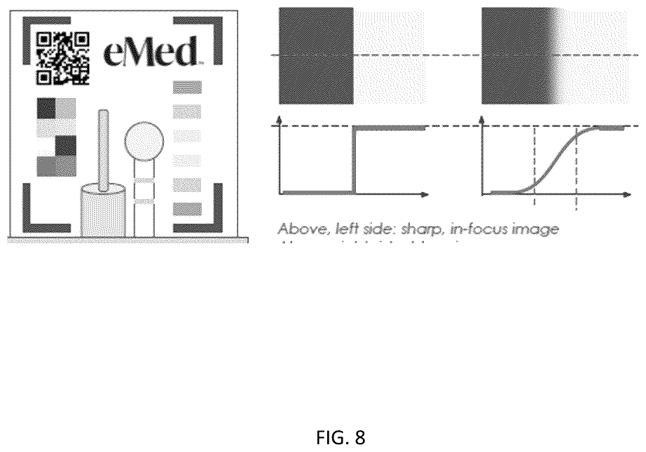

FIG. 8 illustrates an example embodiment of the sharpness of a captured image of a reference card plotted to determine whether the image sharpness is sufficient. The amount of blur in an image can be represented by the “rise time” in pixels, shown by the vertical solid line.

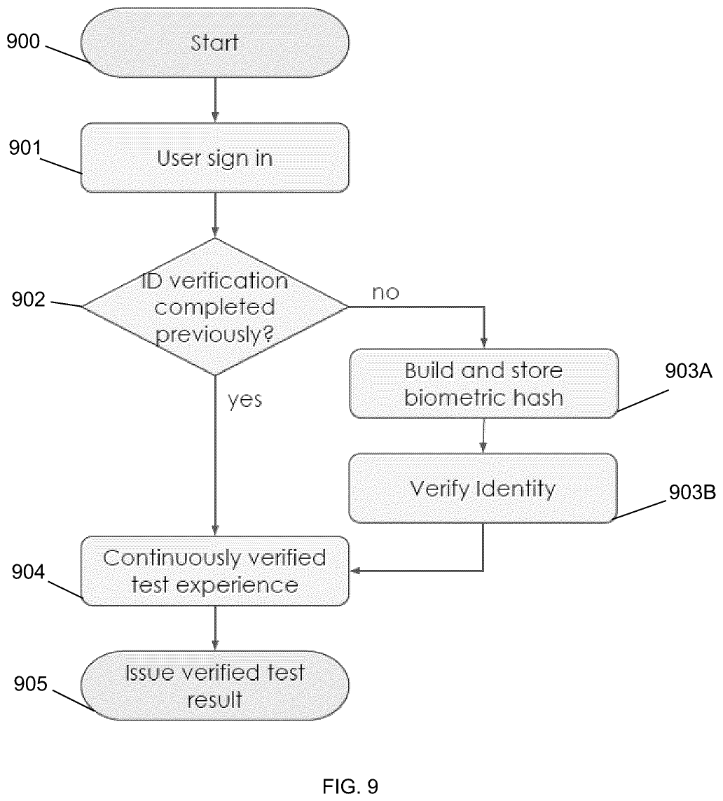

FIG. 9 is a block diagram illustrating an example test process using absolute identity verification. In this approach, a biometric hash is built and stored only once and is thereafter associated with a given user's account along with verification of their government issued ID. After a user starts 900 the testing process, they may be prompted to sign in 901. After sign in 901, the system checks to see if there is a stored biometric hash associated with that particular user at step 902. If the system determines that there is no stored biometric hash for the user, it will prompt the user to build and store a biometric hash in step 903A. After the biometric hash has been created in step 903A, the system will prompt the user to verify their identity in step 903B. A user may verify their identity in step 103B in a variety of ways, depending on requirements. In a non-limiting example, the system may prompt the user to show their face and government-issued ID to a real person (e.g., over a video call) who can then certify the user's identity and complete the one-time verification. In another non-limiting example, the system may verify the user's identity using a third-party verification service or Know Your Customer (KYC). After the user's identity is verified in step 903B, they may proceed to the continuously verified test experience 904. If at step 902, the system determines that there is a stored biometric hash in the system, the user may proceed directly to the continuously verified test experience 904. Video and audio data of the user is continuously captured during the continuously verified test experience 904 and compared to the stored biometric hash to ensure that the same user is present throughout the duration of the test. Once the continuously verified test experience 904 is complete, the system may issue a verified test result 905 for that user.

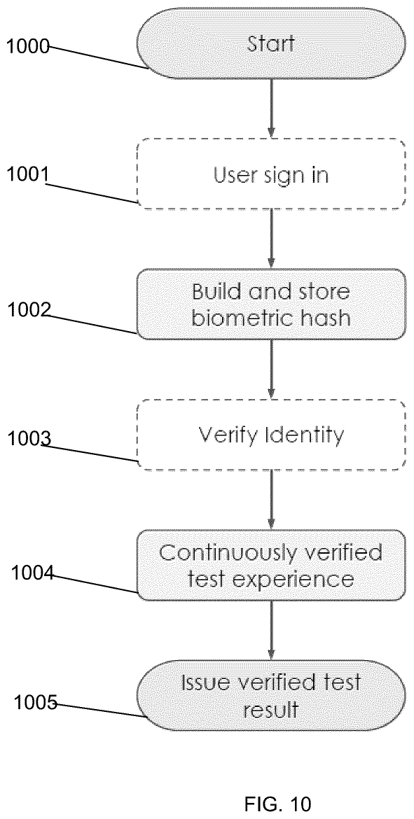

FIG. 10 is a block diagram illustrating an example test process using relative identity verification. In this approach, a biometric hash is built and stored every time any user takes a test. The biometric hash is associated with the test record, as opposed to a given user's account. In test processes using relative identity verification, the steps of signing in and identity verification (e.g., by showing government-issued ID) are optional since they are not required for providing proof-of-result. This is an advantage of test processes using relative identity verification as compared to test processes using absolute identity verification.

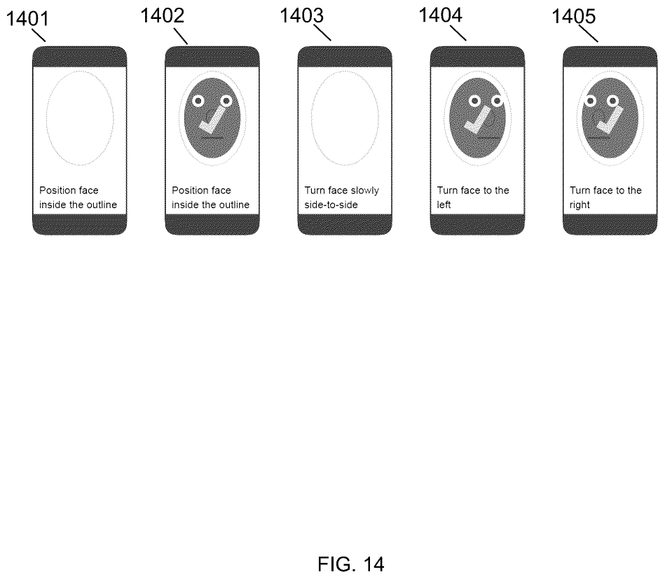

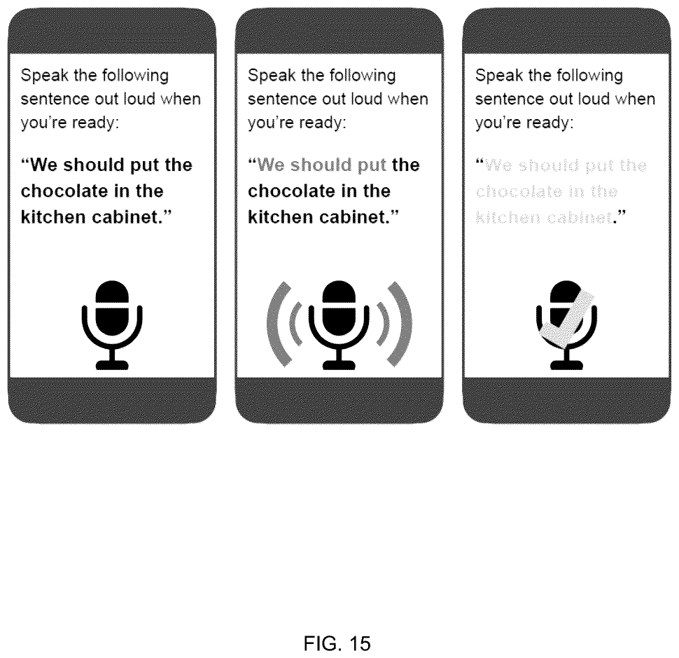

After a user starts 1000 the testing process, they may or may not be prompted to sign in 1001. The user is then prompted to create their biometric hash by recording a video of their face and speaking a provided sentence, as described in FIGS. 14-15. The system uses the video and audio data to build and store the user's biometric hash 202. The system may or may not require the user to verify their identity 1003 after the biometric hash is created. During the continuously verified test experience 1004, face and voice data are captured and analyzed in a rolling window against the stored biometric hash to monitor for irregularities. Once the test result is issued 1005, the user can access the result by using their face and voice, effectively bypassing the need for any ID or document-based verification or account sign in procedures.

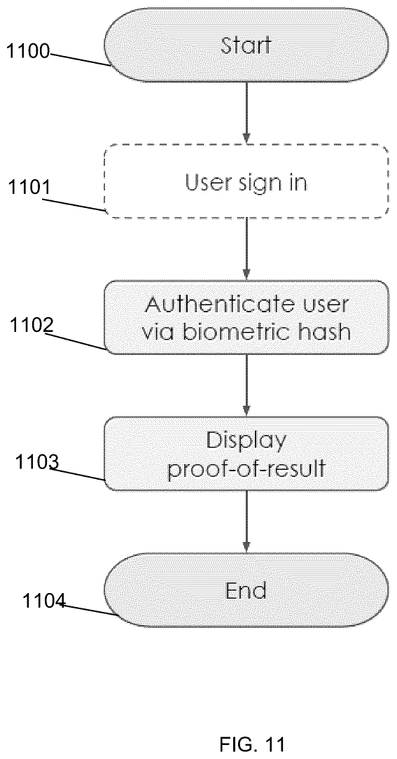

FIG. 11 is a block diagram illustrating an example method for showing verified proof of results using a biometric hash, after a user has already taken a test (e.g., using an absolute or relative identity verification process) and received a verified test result. The biometric hash can be used as a type of credential to unlock the verified result. In a non-limiting example, the method can be implemented on a mobile device application with video and audio recording capabilities. After the user starts 1100 the application, they may or may not be prompted to sign in 1101. After start 1100 and/or log in 1101, the system will then authenticate the user via their biometric hash. The user will be prompted to capture a short video and audio recording of their face and voice, which is compared against their previously stored biometric hash in order to authenticate them as the same person who previously took the test. If the video and audio data from the user's recording matches the stored biometric hash, the authentication is complete 1104 and the user is able to display the proof-of-result for a limited time to a third party. Sign in 1101 is optional in this case since the biometric hash acts as the sign in credentials; however, it may be useful as an additional layer of security or as a portal into past test results and other features.

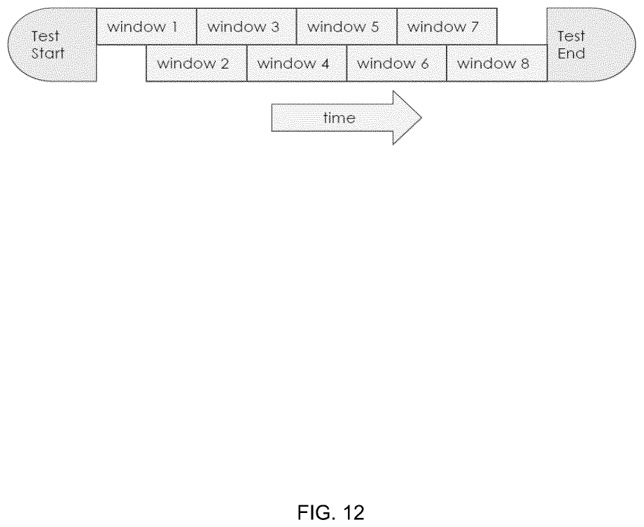

FIG. 12 is a diagram illustrating an example of how the duration of a test can be divided into multiple overlapping time windows. This non-limiting example shows eight windows with a time overlap of 50%. In other embodiments, a different number of windows or different amount of overlap may be preferred depending on the type of test or procedure. For example, long experiences may use long windows with little overlap to reduce the data storage and processing required, while short experiences may use short windows with more overlap to guarantee sufficient data to assess user continuity.

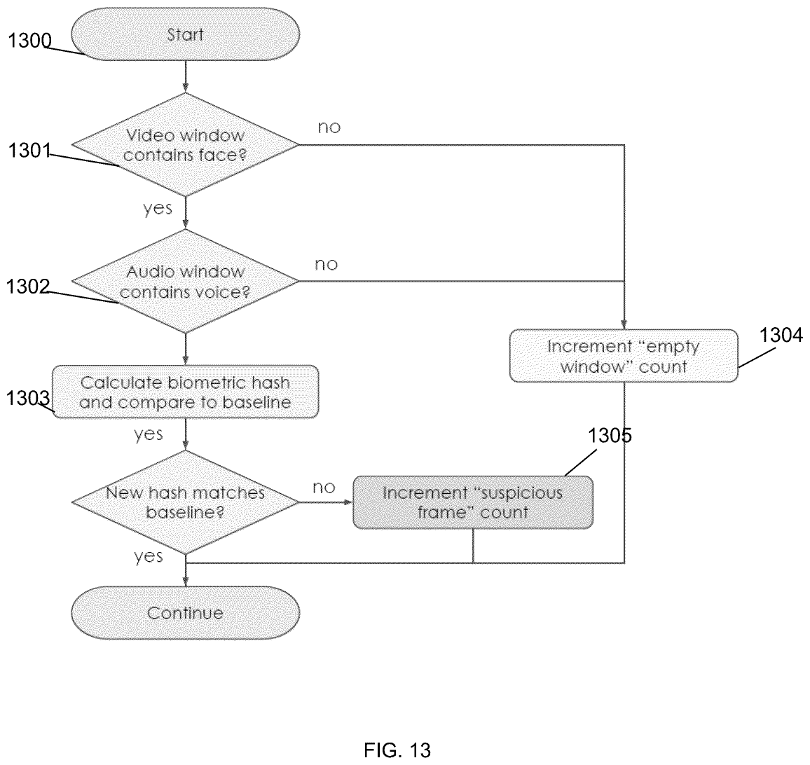

FIG. 13 is a block diagram illustrating an example of a per-window algorithm in operation during a continuously verified test experience. In this non-limiting example, the algorithm and test are implemented on a mobile device with video and audio recording capabilities. After test start 1300, the system will detect if a human face 1301 and voice sample 1302 are found in the given time window. If both a human face and voice sample are detected, a biometric hash is created and compared to the baseline 1303. If it matches, the person can be considered to have remained the same, and no action is taken. If either a human face or voice sample is not found, the algorithm will increment an “empty window” counter 1304. Each test may have a minimum amount of non-empty windows to be considered valid, as the user is expected to remain in front of the camera and to speak a few times during a test. If the empty window counter is too high at the end of the test, it is a signal that that test needs review. If the calculated hash for the window does not match the baseline, the algorithm will increment a “suspicious frame” counter 1305. An increment of the suspicious frame counter could indicate an accomplice was substituted, or other suspicious behavior that may signal the test should be marked and reviewed further for authenticity.

FIG. 14 illustrates an example of a self-guided method for capturing facial recognition data for a biometric hash. In this non-limiting example, a user device (e.g., smartphone, tablet, laptop, desktop, etc.) is used to capture the video frames needed for the biometric hash in only a few seconds. Video recording can begin as soon as the user starts creating the video data for the biometric hash. The user can be prompted to take a video of their face, turning head their slowly to ensure that the front and both sides of their face are captured. First, an outline can appear on the screen and the user can be prompted to, for example, “Position face inside the outline” 1401, in order to ensure that a complete image of the face will be captured. Once the system verifies that a human face is inside the outline displayed on the screen 1402, the system can prompt the user to, for example, “Turn face slowly side-to-side” 1403. A video with the head moving can be important because it ensures that a live video is being captured and that an image of a user's face cannot be substituted for their actual face. Further, requiring the user to turn their face can allow for a more robust model to be created, resulting in a more unique hash. Once the system has detected a video recording of the complete left side of the user's face 1404 and a video recording of the complete right side of the user's face 1405, video recording may stop.

FIG. 15 illustrates an example of a self-guided method for capturing voice recognition data for a biometric hash. In this non-limiting example, a user device (e.g., a smartphone, tablet, laptop, desktop, etc.) is used to capture the audio samples needed for the biometric hash. Audio recording can begin as soon as the user starts creating the audio data for the biometric hash. The system can display a sentence on the screen of the mobile device and can prompt the user to speak the sentence out loud. In s embodiments, the sentence is randomly generated from a set of allowed words to make sure pre-recorded answers can't be used. The sentence may be in any language the test is being completed in. The sentence may include a diversity of sounds to allow a more robust model to be built, resulting in a more unique hash. The sentence may be as short as possible while still including the requisite sound diversity. The system records the audio data and runs it through an algorithm with the video data to create the user's unique biometric hash.

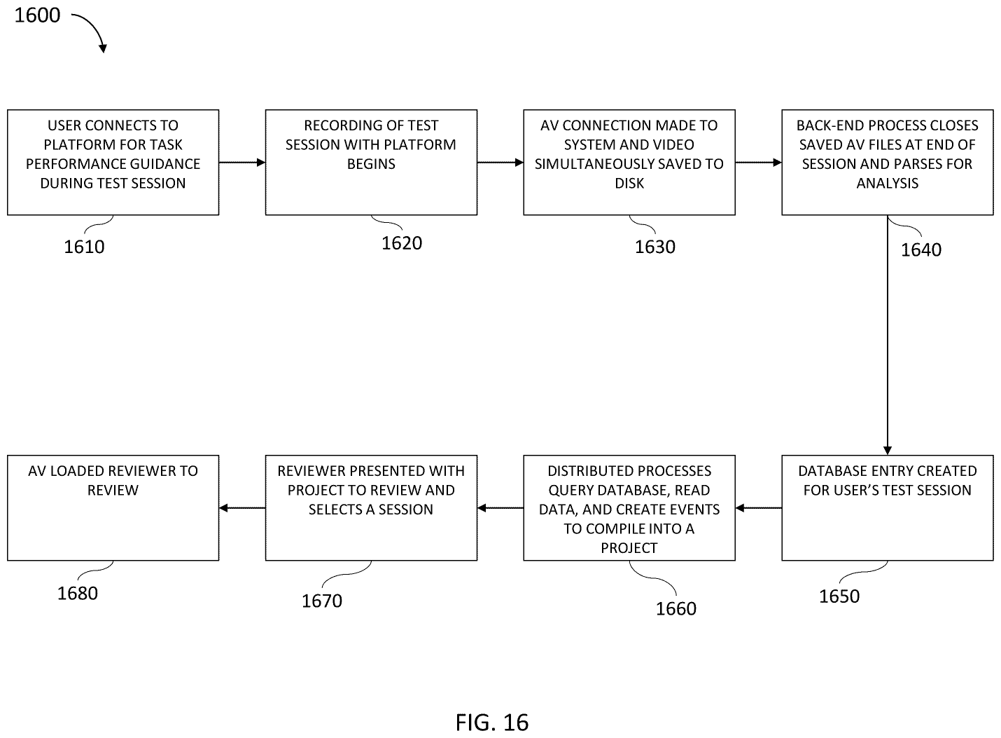

FIG. 16 illustrates a block diagram of an example protocol or method 1600 for a system that event tags and reviews adaptive video parsing. The method 1600 can be implemented, for example, using one or more components of the system shown in FIG. 20.

At block 1610, a user may connect to a service or platform for task performance guidance. The task may include the administration of a health or diagnostic test. The guidance may be provided by a proctor. At block 1620, the system may record the test session to capture the audio, video, or audio and video data of the user and the proctor during the test session. Simultaneously, at block 1630, the audio, video, or audio and video connection may be made to the system and the system may begin saving the data to a disk.

At block 1640, a back-end process within the system may close the saved data files at the end of the test session and parse the data for analysis. The system may parse the data by breaking it into smaller chunks to be analyzed at a later time. At block 1650, a database entry may be created for each user's test session. At block 1660, a configured set of distributed processes may query the database for accessible session and begin reading the data and creating events. The events may also be created while the test session is live or ongoing from the user's browser. The results from the query may be compiled into a project. The project may include more than one test session.

At block 1670, a reviewer may be presented with the project to review and then select which test session to begin with within the project. At block 180, the test session data may be loaded for the reviewer to review.

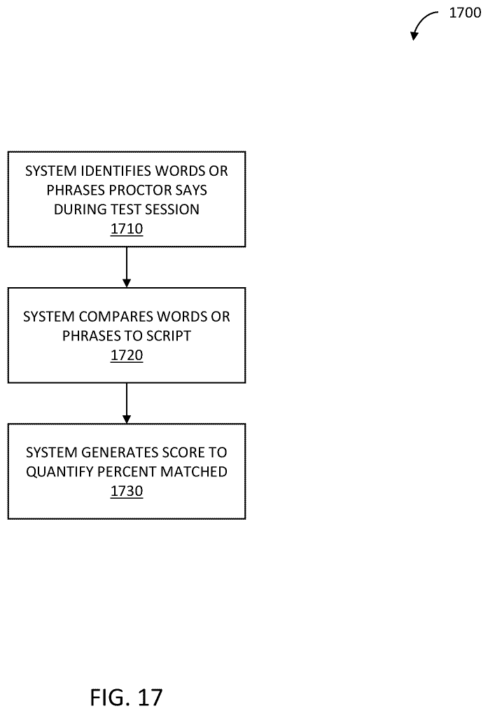

FIG. 17 illustrates a block diagram of an example protocol or method 1700 for a system that evaluates proctor script compliance. The method 1700 can be implemented, for example, using one or more components of the system shown in FIG. 20.

At block 1710, the system may identify words or phrases the proctor says during a test session. These words or phrases can include instructions, introductions, explanations, etc. At block 1720, the system may compare the words or phrases identified to a script that the proctor may have been provided with prior to the start of the test session.

At block 1730, the system may generate a score that may quantify the percent match between the words or phrases identified and the script.

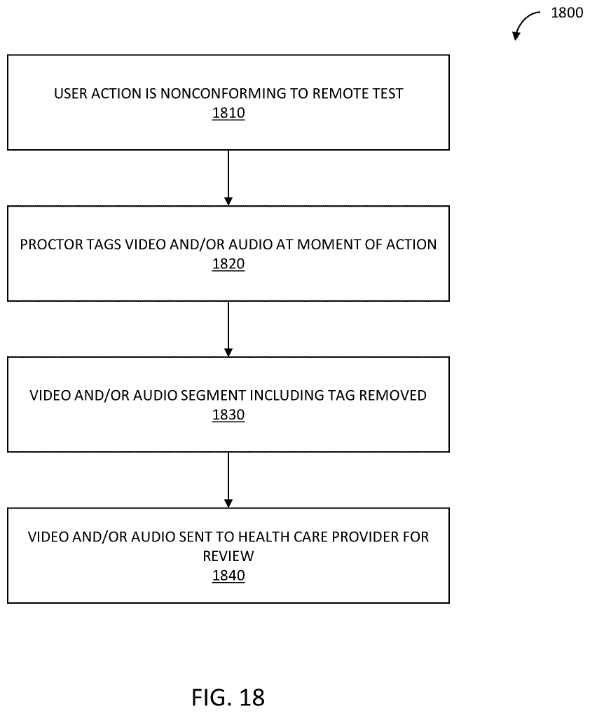

FIG. 18 illustrates a block diagram of an example protocol or method 300 for a system that allows a proctor to tag the video and/or audio produced from a remote health or diagnostic test. At block 1810, a user may act in a nonconforming way to the remote health or diagnostic test. This action may include a suspicious activity, adding an unknown liquid to the diagnostic test, coughing, sneezing, blinking, using emotional words, using a suspicious tone, having more than one face captured in the video, having more than one voice captured in the audio, etc.

At block 1820, a proctor may tag the video and/or audio produced from the remote health or diagnostic test. The proctor may tag the video and/or audio at the moment the proctor identifies the action by the user. At block 1830, the video and/or audio segment that includes the tag may be removed or extracted from the overall video and/or audio produced from the test session. At block 1840, the edited video and/or audio produced from the test session may be sent to a clinician or healthcare provider for review.

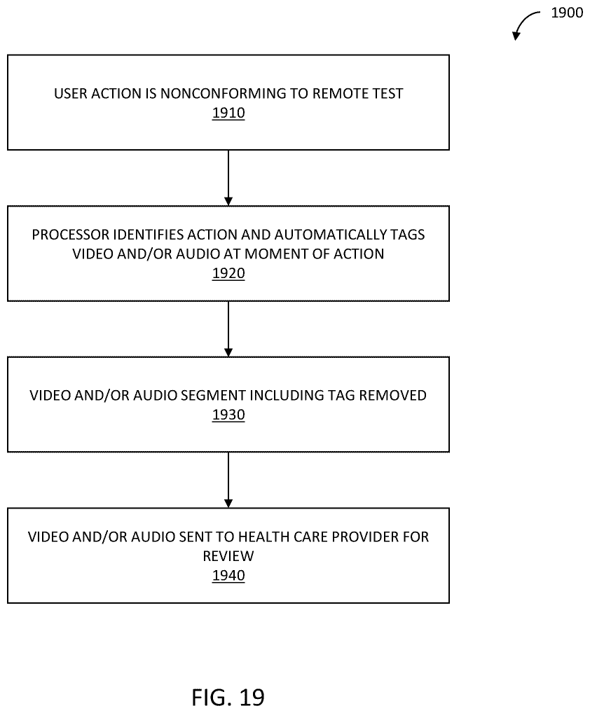

FIG. 19 illustrates a block diagram of an example protocol or method 1900 for a system that allows a proctor to tag the video and/or audio produced from a remote health or diagnostic test. At block 1910, a user may act in a nonconforming way to the remote health or diagnostic test. This action may include a suspicious activity, adding an unknown liquid to the diagnostic test, coughing, sneezing, blinking, using emotional words, using a suspicious tone, having more than one face captured in the video, having more than one voice captured in the audio, etc.

At block 1920, a processor may identify the nonconforming action of the user and automatically tag the video and/or audio produced from the remote health or diagnostic test. The processor may tag the video and/or audio at the moment the proctor identifies the action by the user. At block 1930, the video and/or audio segment that includes the tag may be removed or extracted from the overall video and/or audio produced from the test session. At block 1940, the edited video and/or audio produced from the test session may be sent to a clinician or healthcare provider for review.

FIG. 20 is a block diagram depicting an embodiment of a computer hardware system configured to run software for implementing one or more embodiments of the health testing and diagnostic systems, methods, and devices disclosed herein.

In some embodiments, the systems, processes, and methods described herein are implemented using a computing system, such as the one illustrated in FIG. 20. The example computer system 2002 is in communication with one or more computing systems 2020 and/or one or more data sources 2022 via one or more networks 2018. While FIG. 20 illustrates an embodiment of a computing system 2002, it is recognized that the functionality provided for in the components and modules of computer system 2002 may be combined into fewer components and modules, or further separated into additional components and modules.

The computer system 2002 can comprise a module 2014 that carries out the functions, methods, acts, and/or processes described herein. The module 2014 is executed on the computer system 2002 by a central processing unit 2006 discussed further below.

In general, the word “module,” as used herein, refers to logic embodied in hardware or firmware or to a collection of software instructions, having entry and exit points. Modules are written in a program language, such as JAVA, C or C++, PYPHON or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 2002 includes one or more processing units (CPU) 2006, which may comprise a microprocessor. The computer system 2002 further includes a physical memory 2010, such as random-access memory (RAM) for temporary storage of information, a read only memory (ROM) for permanent storage of information, and a mass storage device 2004, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 2002 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 2002 includes one or more input/output (I/O) devices and interfaces 2012, such as a keyboard, mouse, touch pad, and printer. The I/O devices and interfaces 2012 can include one or more display devices, such as a monitor, that allows the visual presentation of data to a user. More particularly, a display device provides for the presentation of GUIs as application software data, and multi-media presentations, for example. The I/O devices and interfaces 2012 can also provide a communications interface to various external devices. The computer system 2002 may comprise one or more multi-media devices 2008, such as speakers, video cards, graphics accelerators, and microphones, for example.

The computer system 2002 may run on a variety of computing devices, such as a server, a Windows server, a Structure Query Language server, a Unix Server, a personal computer, a laptop computer, and so forth. In other embodiments, the computer system 2002 may run on a cluster computer system, a mainframe computer system and/or other computing system suitable for controlling and/or communicating with large databases, performing high volume transaction processing, and generating reports from large databases. The computing system 2002 is generally controlled and coordinated by an operating system software, such as z/OS, Windows, Linux, UNIX, BSD, SunOS, Solaris, MacOS, or other compatible operating systems, including proprietary operating systems. Operating systems control and schedule computer processes for execution, perform memory management, provide file system, networking, and I/O services, and provide a user interface, such as a graphical user interface (GUI), among other things.

The computer system 2002 illustrated in FIG. 20 is coupled to a network 2018, such as a LAN, WAN, or the Internet via a communication link 2016 (wired, wireless, or a combination thereof). Network 2018 communicates with various computing devices and/or other electronic devices. Network 2018 is communicating with one or more computing systems 2020 and one or more data sources 2022. The module 2014 may access or may be accessed by computing systems 2020 and/or data sources 2022 through a web-enabled user access point. Connections may be a direct physical connection, a virtual connection, and other connection type. The web-enabled user access point may comprise a browser module that uses text, graphics, audio, video, and other media to present data and to allow interaction with data via the network 2018.

Access to the module 2014 of the computer system 2002 by computing systems 2020 and/or by data sources 2022 may be through a web-enabled user access point such as the computing systems' 2020 or data source's 2022 personal computer, cellular phone, smartphone, laptop, tablet computer, e-reader device, audio player, or another device capable of connecting to the network 2018. Such a device may have a browser module that is implemented as a module that uses text, graphics, audio, video, and other media to present data and to allow interaction with data via the network 2018.

The output module may be implemented as a combination of an all-points addressable display such as a cathode ray tube (CRT), a liquid crystal display (LCD), a plasma display, or other types and/or combinations of displays. The output module may be implemented to communicate with input devices 2012 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 2002 may comprise a physical or logical connection established between a remote microprocessor and a mainframe host computer for the express purpose of uploading, downloading, or viewing interactive data and databases on-line in real time. The remote microprocessor may be operated by an entity operating the computer system 2002, including the client server systems or the main server system, an/or may be operated by one or more of the data sources 2022 and/or one or more of the computing systems 2020. In some embodiments, terminal emulation software may be used on the microprocessor for participating in the micro-mainframe link.

In some embodiments, computing systems 2020 who are internal to an entity operating the computer system 2002 may access the module 2014 internally as an application or process run by the CPU 2006.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 2002 may include one or more internal and/or external data sources (for example, data sources 2022). In some embodiments, one or more of the data repositories and the data sources described above may be implemented using a relational database, such as DB2, Sybase, Oracle, CodeBase, and Microsoft® SQL Server as well as other types of databases such as a flat-file database, an entity relationship database, and object-oriented database, and/or a record-based database.

The computer system 2002 may also access one or more databases 2022. The databases 2022 may be stored in a database or data repository. The computer system 2002 may access the one or more databases 2022 through a network 2018 or may directly access the database or data repository through I/O devices and interfaces 2012. The data repository storing the one or more databases 2022 may reside within the computer system 2002.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than restrictive sense.

Indeed, although this invention has been disclosed in the context of certain embodiments and examples, it will be understood by those skilled in the art that the invention extends beyond the specifically disclosed embodiments to other alternative embodiments and/or uses of the invention and obvious modifications and equivalents thereof. In addition, while several variations of the embodiments of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the embodiments of the disclosed invention. Any methods disclosed herein need not be performed in the order recited. Thus, it is intended that the scope of the invention herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and subcombinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination. No single feature or group of features is necessary or indispensable to each and every embodiment.

It will also be appreciated that conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (e.g., as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein, but are to be accorded the widest scope consistent with this disclosure, the principles and the novel features disclosed herein.

What is claimed is:

1. A computer-implemented method for providing an augmented reality user experience to a user comprising:
receiving, from a user device of the user by a computing system, a request for an augmented reality user experience;
generating, by the computing system, data to cause the user device to display the augmented reality user experience on a display of the user device;
transmitting, by the computing system, the data to the user device;
timestamping, by the computing system, the augmented reality user experience based on at least one time interval;
detecting, by the computing system, a failure of the augmented reality user experience;
determining, by the computing system, a timestamp of the failure of the augmented reality user experience;
providing, by the computing system to the user device, instructions to stop the augmented reality user experience; and
providing, by the computing system to the user device, instructions to cause the user device to provide an alternative user experience to the user on the display of the user device.

2. The computer-implemented method of claim 1, further comprising:
providing, by the computing system to the user device, a timestamp of the alternative user experience, the timestamp indicating a timestamp of the alternative user experience at which the user device begins the alternative user experience.

3. The computer-implemented method of claim 2, wherein the timestamp of the alternative user experience is approximately equal to the timestamp of the failure of the augmented reality user experience.

4. The computer-implemented method of claim 2, wherein the timestamp of the alternative user experience is less than the timestamp of the failure of the augmented reality user experience.

5. The computer-implemented method of claim 1, wherein detecting the failure of the augmented reality user experience detecting one or more of: a slowdown in network connection, a reduced frame rate, a loss of object tracking, or a frozen frame.

6. The computer-implemented method of claim 1, wherein the augmented reality user experience comprises a remote health diagnostic exam.

7. The computer-implemented method of claim 1, wherein the at least one time interval is a predetermined time interval.

8. The computer-implemented method of claim 1, wherein the alternative user experience comprises a pre-recorded video.

9. The computer-implemented method of claim 8, further comprises:
generating, by the computing system, the pre-recorded video, wherein the pre-recorded video is based at least in part on an image of an environment of the user capturing using a camera of the user device of the user.

10. The computer-implemented method of claim 1, further comprising:
receiving, by the computing system from the user device, an image frame; and
determining, by the computing system, that the image frame passes a quality threshold.

11. A computer-implemented system for providing an augmented reality user experience to a user comprising:
a non-volatile electronic storage medium, the non-volatile electronic storage medium comprising computer-executable instructions; and
one or more processors in electronic communication with the non-volatile storage medium and configured to execute the computer-executable instructions to cause the computer-implemented system to perform steps comprising:
receiving, from a user device of the user, a request for an augmented reality user experience;
generating, data to cause the user device to display the augmented reality user experience on a display of the user device;
transmitting the data to the user device;
timestamping the augmented reality user experience based on at least one time interval;
detecting a failure of the augmented reality user experience;
determining a timestamp of the failure of the augmented reality user experience;
providing, to the user device, instructions to stop the augmented reality user experience; and
providing, to the user device, instructions to cause the user device to provide an alternative user experience to the user on the display of the user device.

12. The computer-implemented system of claim 11, wherein the computer-executable instructions further cause the computer-implemented system to:
provide, to the user device, a timestamp of the alternative user experience, the timestamp indicating a timestamp of the alternative user experience at which the user device begins the alternative user experience.

13. The computer-implemented system of claim 12, wherein the timestamp of the alternative user experience is approximately equal to the timestamp of the failure of the augmented reality user experience.

14. The computer-implemented system of claim 12, wherein the timestamp of the alternative user experience is less than the timestamp of the failure of the augmented reality user experience.

15. The computer-implemented system of claim 11, wherein detecting the failure of the augmented reality user experience detecting one or more of: a slowdown in network connection, a reduced frame rate, a loss of object tracking, or a frozen frame.

16. The computer-implemented system of claim 11, wherein the augmented reality user experience comprises a remote health diagnostic exam.

17. The computer-implemented system of claim 11, wherein the at least one time interval is a predetermined time interval.

18. The computer-implemented system of claim 11, wherein the alternative user experience comprises a pre-recorded video.

19. The computer-implemented system of claim 18, wherein the computer-executable instructions further cause the computer-implemented system to:
generate the pre-recorded video, wherein the pre-recorded video is based at least in part on an image of an environment of the user capturing using a camera of the user device of the user.

20. The computer-implemented system of claim 11, wherein the computer-executable instructions further cause the computer-implemented system to:
receive, from the user device, an image frame; and
determine, that the image frame passes a quality threshold.
